(19) United States
(12) Patent Application Publication
(10) Pub. No.: US 2020/0179286 A1
(43) Pub. Date: Jun. 11, 2020

(54) CARRIER STRUCTURE AND DRUG CARRIER, AND PREPARING METHODS THEREOF

(71) Applicant: GENE'E TECH CO., LTD., New Taipei (TW)

(72) Inventors: Shu-Jyuan Yang, New Taipei (TW); Chung-Hao Wang, New Taipei (TW)

(21) Appl. No.: 16/789,443

(22) Filed: Feb. 13, 2020

Related U.S. Application Data
(63) Continuation-in-part of application No. PCT/CN2018/074590, filed on Jan. 30, 2018.

(30) Foreign Application Priority Data
Jan. 19, 2018 (CN) 201810053594.0

(51) Int. Cl. A61K 9/16; A61K 31/4439; A61K 31/43; A61K 31/7048

(52) U.S. Cl. CPC A61K 9/1652; A61K 9/1629; A61K 31/7048; A61K 31/4439; A61K 31/43; A61K 9/1617

(57) ABSTRACT

A drug carrier carrying an active substance and a method preparing the same are provided. The method includes respectively dissolving a negatively charged polymer, sodium tripolyphosphate, and an active substance in a NaOH aqueous solution to increase the encapsulation rate of the active substance in the drug carrier.

### CROSS REFERENCE TO RELATED APPLICATIONS

The present application is a Continuation-in-part Application of PCT Application No. PCT/CN2018/074590 filed on Jan. 30, 2018, which claims the benefit of Chinese patent applicatin No. 201810053594.0 filed on Jan. 19, 2018. All contents of the above applications are incorporated herein by reference in their entirety.

### FIELD

The disclosure relates to a drug-carrying technology, especially relates to a drug carrier carrying an active substance.

### BACKGROUND

In a human body, gastric acid and pepsin secreted by the gastro could not only decompose and digest food but also remove harmful bacteria that enter through the mouth. However, after the human body is infected by Helicobacter pylori (H. pylori), H. pylori could convert urea into alkaline ammonia through the secretion of urease in order to avoid being destroyed by gastric acid. When the body's immune system is against bacteria, the stomach protection mechanism would be damaged by chronic inflammation, and further cause chronic inflammation or peptic ulcer (gastric or duodenal wall damage). Without proper treatment, complications such as gastrointestinal bleeding, perforation, or obstruction of the outlet could result. In the worst cases, it could cause gastric cancer. The infection rates of H. pylori are 100% in chronic gastritis, is 90-95% in duodenal ulcer, 60-80% in gastric ulcer, 80% in gastric lymphoma, and 90% in gastric cancer, respectively.

In general, the infection rate of H. pylori increases with age and its prevalence varies slightly from place to place, with nearly all adults in developing countries carry the bacterium (about 90%), but infection rates in developed countries are much lower (about 11%).

Eradication of H. pylori is mainly treated with so-called “triple therapy” or “quadruple therapy,” which is a combination of proton pump inhibitors and antibiotics. However, due to the long course of eradication treatment of H. pylori, the number of drugs taken in for a single time is up to about 10 tablets, and the eradication of pylori drugs often cause patients having dizziness, diarrhea, tongue coating, taste sense dulling, allergy and other side effects, so that the patient's compliance is low, leading to treatment failure.

Among the present technologies, a technique containing cross-linked polyglucosamine and amoxicillin nanosomes is disclosed. In this technique, an anionic surfactant and oil were mixed to form a water-in-oil emulsion to encapsulate amoxicillin by cross-linked polyglucosamine. In this technique, the average particle diameter is 100-600 nm and the encapsulated amoxicillin is at least 5% (w/w) of the total weight of the nanosomes. When taking orally, the nanosomes have a longer retention time in the stomach than free amoxicillin or micron-sized particles.

In addition, a technique of shell-core drug structure also exists. The shell-core drug structure is formed by encapsulating a drug with alginate as a matrix to form microspheres and then encapsulating the microspheres with an outer membrane of chitosan to form the shell-core drug structure. The drug can be sustained released by the colloidal alginate. Such a drug structure may help protect drugs from being damaged in stomach acid, but when used in the treatment of stomach ulcers, it does not address the drawback of having to use multiple drugs.

Furthermore, a drug structure including alginate and chitosan is present, in which calcium pantothenate is added to induce sodium alginate to form colloidal particles to encapsulate the drug. The drug structure has a property of releasing the drug within 2 hours, but the drug structure still fails to address the disadvantage of having to use multiple drugs in the clinical treatment of gastric ulcers.

To sum up, improving drug structure may be one of the feasible strategies to break the bottleneck of clinical treatment of gastric ulcer. All the drugs shown in the above-mentioned technologies involve the use of alginate and chitosan, but the efficacy characteristics are not the same, and there is still room for improvement. Obviously, although alginate and chitosan preparation of drug carriers is potential materials, the relative proportion of ingredients, combined structure and method, size of the prepared carrier and other parameters are still substantial and significantly affect the efficiency and characteristics of the prepared medical structure. Therefore, the search for the ratio and method with the best effect is the most critical and creative feature of the related technology.

### SUMMARY

In light of the foregoing, the disclosure provides a method of preparing a carrier structure for carrying a drug. The method comprises the following steps. An aqueous solution of a negatively charged polymer with a pH value of 6-8 and an aqueous solution of sodium tripolyphosphate with a pH value of 6-8 are prepared by respectively dissolving the negatively charged polymer and the sodium tripolyphosphate in a NaOH aqueous solution. An aqueous solution of chitosan is prepared with a pH value of 3-5. 100 parts by weight of the aqueous solution of the negatively charged polymer, 330-1000 parts by weight of the aqueous solution of the sodium tripolyphosphate, and 830-2500 parts by weight of the aqueous solution of chitosan are mixed to form an initial mixture. The initial mixture is reacted for 5-60 minutes to self-assemble the negatively charged polymer, the sodium tripolyphosphate, and the chitosan to form the drug carrier.

In one embodiment, the carrier structure has a diameter of 90-150 nm.

In another embodiment, the carrier structure in an aqueous solution has a surface potential of is 15-30 mV.

In yet another embodiment, the negatively charged polymer comprises alginate, heparin, polyacrylic acid, polystyrene sulfonate, poly(maleic acid), hyaluronic acid, or any combinations thereof.

In the carrier structure for carrying a drug prepared by the method above, the components that not only have excellent biocompatibility but also contribute to the release of the drug or the residence time of the drug in the body, thus enhancing the efficacy of the drug.

In addition, the disclosure also provides a method of preparing a drug carrier carrying an active substance, and the method comprises the following steps. An aqueous solution of a negatively charged polymer with a pH value of 6-8, an aqueous solution of sodium tripolyphosphate with a pH value of 6-8, and an aqueous solution of the active substance with a pH value of 6-8 are prepared by respectively dissolving the negatively charged polymer, the sodium tripolyphosphate, and the active substance in an aqueous solution of NaOH. 100 parts by weight of the aqueous solution of the negatively charged polymer, 330-1000 parts by weight of the aqueous solution of the sodium tripolyphosphate, and 2000-3000 parts by weight of the aqueous solution of the active substance are mixed to form an initial mixture. 830-2500 parts by weight of an aqueous solution of chitosan with a pH value of 3-5 is added into the initial mixture to form an active mixture. The active mixture is reacted for 5-60 minutes to self-assemble the negatively charged polymer, the sodium tripolyphosphate, the active substance and the chitosan to form the drug carrier carrying the active substance.

In one embodiment, the drug carrier has a particle diameter of 110 to 160 nm.

In another embodiment, the drug carrier in an aqueous solution has a surface potential of 15-25 mV.

In still another embodiment, the active substance inhibits the activity of H. pylori.

In yet still another embodiment, the active substance comprises amoxicillin, clarithromycin, omeprazole, penicillin, or any combinations thereof.

In yet still another embodiment, the negatively charged polymer comprises alginate, heparin, polyacrylic acid, polystyrene sulfonate, poly(maleic acid), hyaluronic acid, or a combination thereof.

In yet still another embodiment, an encapsulation rate of the active substance in the drug carrier is 55-75%.

In yet still another embodiment, the drug carrier carrying the active substance contains 32-38 wt % of the active substance.

For the drug carrier carrying an active substance prepared by the method above, the effect of the drug is more fully developed and has a better curative effect through the design of the component proportion and solvent selection. Moreover, the above method of preparing the drug carrier carrying an active substance is not only simple but also has a higher drug encapsulating rate, stable particle size and surface potential, and higher biocompatibility.

Furthermore, a method of treating a gastrointestinal disease is provided. The method comprises applying an effective dosage of the above drug carrier carrying an active substance to a host having a gastrointestinal disease caused by Helicobacter pylori. The active substance comprises amoxicillin, clarithromycin, omeprazole, penicillin or any combinations thereof.

In one embodiment, the effective dosage is 1-10 mg/kg body weight per day.

In another embodiment, the host is a human

In yet another embodiment, the drug carrier carrying an active substance further comprises an adjuvant, an excipient, a medically acceptable carrier, or any combinations thereof.

In still another embodiment, the gastrointestinal disease is caused by H. pylori.

In still another embodiment, the gastrointestinal diseases comprise chronic gastritis, duodenal ulcer, gastric ulcer, gastric lymphoma, gastric cancer, gastric mucosal atrophy, intestinal metaplasia or any combinations thereof.

To sum up, the carrier structure of the disclosure and the types and proportions of the components contained in the drug carrier contribute to the release of the active substance in the organism and the extension of its residence time, thus the effect of the drugs could be more complete. In addition, the pharmaceutical composition of the disclosure is designed to allow the contained ingredients to combine with each other by electrostatic attraction, and further achieve a higher encapsulation rate of the active substance. In other words, the pharmaceutical composition of the disclosure is a mixed structure of its components, which is not a shell-core structure, and no anionic surfactant and oil need to be added to form water-in-oil emulsion. Therefore, the preparation method is much easier than the preparation method of conventional drugs with shell-core structure or water-in-oil structure.

Furthermore, when the drug structure of the disclosure adheres to the mucosal tissue and gets close to the neutral environment of the gastric parietal cell layer, the nano-structure of the drug carrier gradually dissolves due to the change of the charged characteristics of chitosan and alginate or polyacrylic acid, which results in the release of the active substances in the drug carrier. The release property allows the drug to be released closer to the pathogen accumulation site, helping to improve the efficacy of the active substance.

### BRIEF DESCRIPTION OF THE DRAWINGS

By referring to the attached drawings and embodiments, the above and other characteristics and advantages would be more apparent for the person skilled in the art.

FIG. 1 depicts a flow chart of a method of preparing a carrier structure for carrying an active substance according to an embodiment of this invention.

FIG. 2 depicts a flow chart of a method of preparing a drug carrier carrying an active substance according to an embodiment of this invention.

FIG. 3 depicts a relation diagram of pH value versus average particle diameter and surface potential of the drug carrier according to some embodiments of this invention.

FIG. 4 are images of the samples in FIG. 3 observed by a transmission electron microscope.

FIG. 5 depicts a relation diagram of pH value versus average particle diameter and surface potential of the drug carrier according to some other embodiments of this invention.

FIG. 6 are images of the samples in FIG. 4 observed by a transmission electron microscope.

FIGS. 7 and 8 depict results of in vitro tests of the drug carrier according to some embodiments of this invention.

FIG. 9 depicts a relation diagram of average particle size versus different pH values of AMO/alginates/soldium tripolyphosphare solution when the nanoparticiles were prepared with chitosan:alginate:sodium tripolyphosphate:amoxicillin weight ratios of 25:1:10:30 and 12.5:1:5:15.

FIG. 10 depicts a diagram of encapsulating rate versus different pH values of AMO/alginates/soldium tripolyphosphare solution when the nanoparticiles were prepared with chitosan:alginate:sodium tripolyphosphate:amoxicillin weight ratios of 25:1:10:30 and 12.5:1:5:15.

### DETAILED DESCRIPTION

A more comprehensive illustration of some embodiments would be provided below with reference to the attached drawings. However, they could be implemented by different forms, and understood without limitations referring to these embodiments. Instead, these embodiments are provided so that the disclosure is complete, and the scope of the disclosure would be fully expressed to persons skilled in the art.

In some embodiments of this invention, the carrier structure and drug carrier are prepared by selecting specific types and proportions of ingredients as well as the mixing sequence. Compared with the present technology, the carrier structure and drug carrier are more conducive to improving the efficacy of the drug. In detail, with the disclosed carrier structure and the drug carrier after the combination with the active substance, H. pylori could be well inhibited by a single drug. Thus, the disclosure would break through the current technical bottleneck of treating gastric ulcer with multiple active substances and hydrogen proton pump inhibitors.

At the microscopic level, the the effect of “inhibiting H. pylori” in the disclosure indicates the capability of “controlling the population of H. pylori,” “reducing the population of H. pylori” and/or “vanishing the population of H. pylori.” At the microscopic level, it indicates the capability of “reducing the physiological effects of H. pylori,” “reducing the infectivity of H. pylori” and/or “killing H. pylori.”

“Substances inhibiting H. pylori” means substances having the effects of “inhibiting H. pylori” mentioned above, such as antibiotics (amoxicillin, clarithromycin and penicillin, etc.). More specifically, the so-called “active substances” in the disclosure could refer to substances that inhibit H. pylori.

“Substances that can assist in inhibiting H. pylori” means the substances that do not directly have “the capability of inhibiting H. pylori” mentioned above, but can contribute to the effect of such substances. More specifically, in addition to triple or quadruple antibiotics in the current administration of gastric ulcers, hydrogen proton pump inhibitors also need to be used. Hydrogen proton pump inhibitors do not directly inhibit the ability of H. pylori, but help to enhance the effectiveness of antibiotics. Specifically, “substances that can assist in inhibiting H. pylori” could be the aforementioned hydrogen proton pump inhibitors, and bismuth agents, etc.

“Substances that could assist in inhibiting H. pylori” exclude substances designed in pharmacology to assist in the administration of drugs, to improve its taste, or to extend the preservation of the drugs. That is, it does not include any additives commonly used in the formulation of drugs, such as medical carrier agents, flavorants, or preservatives.

In the preparation method of this disclosure, chitosan is used. Chitosan is a quite popular natural polymer in recent years. The source of chitosan is usually obtained from performing the deacetylation reaction of chitin by high-concentration hot alkali treatment, so that the acetyl groups of chitosan can be converted to amine groups. Chitosan can be widely used in the pharmaceutical field because of its positive charge and mucoadhesiveness in acidic environment. General business lists of the molecular weight of chitosan molecules is about 3800 to 20000 kDa, deacetylation rate is 66% to 95%. Due to highly reactive groups, such as amine group and hydroxyl group, of chitosan, chitosan can be used to prepare some other derivatives of chitosans and can be dissolved in weakly acidic aqueous solution. Therefore, chitosan could be made into thin films, spheres, fibers, or gels according to the application needs.

The molecular weight of the chitosan used in the disclosure could be 4,000 kDa, 5,000 kDa, 6,000 kDa, 7,000 kDa, 8,000 kDa, 9,000 kDa, 10,000 kDa, 11,000 kDa, 12,000 kDa, 13,000 kDa, 14,000 kDa, 15,000 kDa, 16,000 kDa, 17,000 kDa, 18,000 kDa, 19,000 kDa, 20,000 KDa, or any ranges in between. The deacetylation rate of the chitosan used in the disclosure could be 66%, 68%, 70%, 72%, 74%, 76%, 78%, 80%, 82%, 84%, 86%, 88%, 90%, 92%, 94% or any ranges in between. However, in some embodiments of this invention, the molecular weight of chitosan is about 15,000 kDa, and the deacetylation rate is 84%.

The negatively charged polymer adopted in the disclosure refers to a polymer with negative charges in neutral and acidic environment; for example, polymers with negative charges at a pH of 1 to 8, such as at a pH of 2 to 8. The negatively charged polymers comprise, but are not limited to, alginate, heparin, polyacrylic acid, polystyrene sulfonate, poly(maleic acid), or hyaluronic acid. In some embodiments, the negatively charged polymers may be alginate, polyacrylic acid, or any combinations thereof.

The active substance is all compounds intended to be used for treatment, prevention, and detection. In the disclosure, the active substance may be a compound used for the treatment of gastric ulcer. That is, the active substance has an activity of inhibiting H. pylori and comprises amoxicillin, clarithromycin or penicillin. The pharmaceutical composition in the disclosure could comprise several active substances inhibiting H. pylori. In some embodiments, only a single active substance inhibiting H. pylori is used in the pharmaceutical composition of the disclosure.

In some embodiments, the chitosan, a negatively charged polymer, sodium tripolyphosphate, and/or active substance are in solution state. This would help control the pH values of each component so that they are properly charged.

The technical content of the disclosure is described in detail with embodiments and drawings hereafter; however, the following descriptions are exemplary, and are not used to limit implememntation aspects of the disclosure.

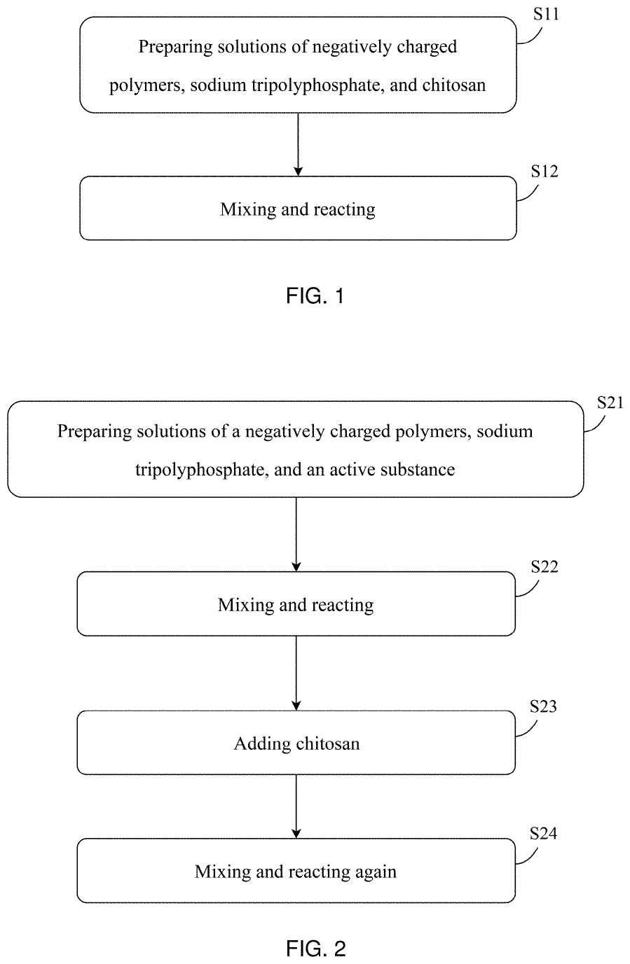

Referring to FIG. 1, FIG. 1 depicts a flow chart of a method of preparing a carrier structure for carrying an active substance according to an embodiment of this invention. In some embodiments, the method of preparing the carrier structure comprises steps S11 to S13. In step S11, 100 parts by weight of negatively charged polymer solution with a pH value of 6-8, 330 to 1000 parts by weight of sodium tripolyphosphate solution with a pH value of 6-8, and 830 to 2500 parts by weight of chitosan with a pH value of 3 to 5 are prepared.

That is, when the negatively charged polymer solution is 100 parts of weight, the sodium tripolyphosphate solution could be 330, 350, 400, 450, 500, 550, 600, 650, 700, 750, 800, 850, 900, 950, 1000 parts by weight or any ranges in between. The chitosan solution could be 830, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, or 2500 parts by weight or any ranges in between.

In some embodiments, the concentration of the negatively charged polymer solution could be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20 mg/ml or any ranges in between; and pH value of the negatively charged polymer solution could be 6.0, 6.2, 6.4, 6.6, 6.8, 7.0, 7.2, 7.4, 7.6, 7.8, 8.0 or any ranges in between. The concentration of the sodium tripolyphosphate solution could be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mg/ml or any ranges in between; the pH value of the sodium tripolyphosphate solution could be 6.0, 6.2, 6.4, 6.6, 6.8, 7.0, 7.2, 7.4, 7.6, 7.8, 8.0 or any ranges in between. The concentration of the chitosan solution could be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mg/ml or a range between; and the pH value of the chitosan solution could be 3.0, 3.2, 3.4, 3.6, 3.8, 4.0, 4.2, 4.4, 4.6, 4.8, 5.0 or any ranges in between.

Next, in step S12, the aforementioned negatively charged polymer solution, sodium tripolyphosphate solution and chitosan solution are mixed to form an initial mixture. After reacting for a while, self-assembled carrier structures are obtained from th initial mixture. In some embodiments, the reaction time could be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes or any ranges in between. In some embodiments, the reaction temperature could be 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C. or any ranges in between.

The carrier structure prepared by the method mentioned above could maintain a stable charged state and a stable structure during preservation. Moreover, the method above does not comprise any step of particle size uniformization or finalization, such as extrusion, etc., so that the carrier structure with required particle size could be directly obtained. In some embodiments, in order to maintain the carrier structure by maintaining the properly charged state of the ingredients within the carrier structure, the carrier structure could be stored and/or used in solution. In some embodiments, when stored and/or used, the solution is adjusted to the appropriate pH value, such as 3.0, 3.5, 4.0, 4.5, 5.0, 5.5, etc. or any ranges in between, to maintain the charged state of the ingredients each more stably.

In the carrier structure of some embodiment, chitosan, the negatively charged polymer and the sodium tripolyphosphate could be combined with each other by electrostatic attraction to self-assemble into particles with specific size and having good biocompatibility. In some embodiments, the particle diameter of the assembled carrier structure could be in nanometer size, such as 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, or any ranges in between. The nanoparticles with the above size are beneficial to the absorption efficiency in vivo and could enhance the function of the carrier structure in carrying drugs.

[60] In addition, the surface potential of the assembled carrier structure surface could be positive, such as 15 mV, 16 mV, 17 mV, 18 mV, 19 mV, 20 mV, 21 mV, 22 mV, 23 mV, 24 mV, 25 mV, 26 mV, 27 mV, 28 mV, 29 mV, 30 mV or any ranges in between. Surface structures with the above range of surface charges contribute to the retention time of the carrier structure in the stomach.

On the other hand, referring to FIG.2, FIG. 2 depicts a flow chart of a method of preparing a drug carrier carrying an active substance according to an embodiment of this invention. In some embodiments, the method of preparing the drug carrier of the disclosure comprise steps S21 through S24. In step S21, 100 parts by weight of negatively charged polymer solution with a pH value of 6-8, 330 to 1000 parts by weight of sodium tripolyphosphate solution with a pH value of 6-8, and 2000 to 3000 parts by weight of an active substance solution with a pH value of 6-8 are prepared. The aqueous solution of the negatively charged polymer, sodium tripolyphosphate and the active substance are mixed.

That is, when the negatively charged polymer solution is 100 parts of weight, the sodium tripolyphosphate solution could be 330, 350, 400, 450, 500, 550, 600, 650, 700, 750, 800, 850, 900, 950, 1000 parts by weight, or any ranges in between; and the active substance solution could be 2000, 2050, 2100, 2150, 2200, 2250, 2300, 2350, 2400, 2450, 2500, 2550, 2600, 2650, 2700, 2750, 2800, 2850, 2900, 2950, 3000 parts by weight, or any ranges in between.

In some embodiments, the concentration of the negatively charged polymer solution could be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20 mg/ml or any ranges in between; and pH value of the negatively charged polymer solution could be 6.0, 6.2, 6.4, 6.6, 6.8, 7.0, 7.2, 7.4, 7.6, 7.8, 8.0 or any ranges in between. The concentration of the sodium tripolyphosphate solution could be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mg/ml or any ranges in between; the pH value of the sodium tripolyphosphate solution could be 6.0, 6.2, 6.4, 6.6, 6.8, 7.0, 7.2, 7.4, 7.6, 7.8, 8.0 or any ranges in between. The concentration of the active substance solution could be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mg/ml or any ranges in between; and the pH value of the active substance solution could be 6.0, 6.2, 6.4, 6.6, 6.8, 7.0, 7.2, 7.4, 7.6, 7.8, 8.0 or any ranges in between.

Next, in step S22, the aforementioned negatively charged solution, sodium tripolyphosphate solution and the active substance solution are mixed and then reacted for a while to form an initial mixture. In some embodiments, the reaction time could be 10, 11, 12, 13, 14, 15, 16, 17, 18, 19, 20, 21, 22, 23, 24, 25 minutes or any ranges in between. In some other embodiments, the reaction temperature could be 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or any ranges in between.

In step S23, 830-2500 parts by weight of chitosan with a pH value of 3-5 is added with the initial mixture to form an active mixture. The active mixture is reacted for 5-60 minutes to self-assemble the negatively charged polymer, sodium tripolyphosphate, active substance and chitosan to form the drug carrier containing the active substance. That is, when the negatively charged polymer solution is 100 parts by weight, the chitosan solution could be 830, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500 parts by weight or any ranges in between. The concentration of the chitosan solution could be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mg/ml or any ranges in between; and the pH value of the chitosan solution could be 3.0, 3.2, 3.4, 3.6, 3.8, 4.0, 4.2, 4.4, 4.6, 4.8, 5.0 or any ranges in between.

In some embodiments, the reaction time after adding chitosan could be 5, 6, 7, 8, 9, 10, 11, 12, 13, 14, 15, 16, 17, 18, 19, 20, 21, 22, 23, 24, 25, 27, 28, 29, 30 minutes or any ranges in between. In some embodiments, the reaction temperature could be 4° C., 5° C., 6° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C. or any ranges in between.

Similarly, the drug carrier prepared by the method aforementioned could maintain a stable charged state and a stable structure when preserved. The method above does not comprise any steps of particle size uniformaliztion or finalization, such as extrusion, etc. to obtain the drug carrier with required particle size. In some embodiments, in order to maintain the drug carrier by maintaining the proper charged state of the ingredients within the drug carrier, the drug carrier could be stored and/or used in solution. In some embodiments, the solution could be stored and/or used at a pH value appropriate to maintain the charged state of the components more stably, such as at a pH value of 3.0, 3.5, 4.0, 4.5, 5.0, 5.5, or any ranges in between.

In the drug carrier of some embodiments, the chitosan, negatively charged polymer, sodium tripolyphosphate and the active substance could be combined with each other by electrostatic attraction and self-assembled into particles with specific size and having good biocompatibility. In some embodiments, the assembled drug carrier could be in nanometer size, such as 100 nm, 105 nm, 110 nm, 115 nm, 120 nm, 125 nm, 130 nm, 135 nm, 140 nm, 145 nm, 150 nm, 155 nm, 160 nm, 165 nm, 170 nm or any ranges in between. The nanoparticles with the above size are beneficial to the absorption efficiency in vivo and could enhance the function of drug carriers.

In addition, the surface potential of the assembled drug carrier surface could be positive. In some embodiments, the surface potential of the assembled drug carrier surface could be 15 mV, 16 mV, 17 mV, 18 mV, 19 mV, 20 mV, 21 mV, 22 mV, 23 mV, 24 mV, 25 mV or any ranges in between. Surface structures with the above range of surface charges contribute to the retention time of the drug carrier in the stomach.

It should be noted that in the preparing method of the carrier structure and the drug carrier, the mixing sequences of the three solutions (i.e. the solutions of the negatively charged polymer, the chitosan and the sodium tripolyphosphate), other than the solution of the active substance solution, could be changed. In some embodiments, in the preparing method of the carrier structure, the negatively charged polymer solution, the chitosan solution and the sodium tripolyphosphate solution could be directly mixed. However, in the preparing method of the drug carrier, the encapsulating rate could be improved by first mixing the negatively charged polymer solution, the sodium tripolyphosphate solution and the active substance solution, and adding and then mixing the chitosan solution.

In the embodiment, the encapsulating rate of the active substance by the drug carrier could be 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, 75 wt % or any ranges in between. The parts by weight of the active substance in the drug carrier could be approximately 30 wt %, 31 wt %, 32 wt %, 33 wt %, 34 wt %, 35 wt %, 36 wt %, 37 wt %, 38 wt %, 39 wt %, 40 wt %, or any ranges in between.

The disclosure also provides a method of treating gastrointestinal diseases by using the drug carrier above. The method comprises preparing the drug carrier in accordance with the preparing method aforementioned and delivering an effective dosage of the drug carrier to H. pylori or its population in a host. In addition, it includes other steps that are not aimed at inhibiting H. pylori, including reducing the number of times of taking drugs, relieving side effects caused by the drug, and helping the individual to rest.

In some embodiments, the effective dosage could be the dosage of the drug carrier that effectively inhibits H. pylori without causing discomforts or side effects to the host. In some embodiments, the effective dosage could be 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 mg/kg/day or any ranges in between. In addition, the effective dosage could be administered several times in one day or once a few days, such as once a day, twice a day, three times a day, once every two days, once every three days, or any ranges in between.

In this disclosure, some examples of the carrier structure and the drug carrier are described below, and their physicochemical properties are analyzed and measured.

In an example of the carrier structure of the disclosure, chitosan was dissolved in 0.01 M acetic acid to achieve the concentration of 0.5 mg/ml and pH=4.0. Alginate or polyacrylic acid was dissolvced in 0.01 N NaOH solution to achieve the concentration of 0.05 mg/ml and pH=7.4. Sodium tripolyphosphate was dissolved in 0.01 N NaOH solution to achieve the concentration of 0.5 mg/ml and pH=7.4. Next, according to steps S11 to S12, Samples 1-7 are prepared by using the proportions listed in Tables 1-2 below. The obtained results of particle size analysis and surface potential analysis of samples 1-7 are shown in Table 3 below.

TABLE 1
Weight ratios of each components used in samples 1-4.

| Sample | Chitosan | Alginate | Sodium Tripolyphosphate |
|---|---|---|---|
| 1 | 25 | 1 | 10 |
| 2 | 16.7 | 1 | 6.7 |
| 3 | 12.5 | 1 | 5 |
| 4 | 8.3 | 1 | 3.3 |

TABLE 2
Weight ratios of each components used in samples 5-7.

| Sample | Chitosan | Polyacrylic Acid | Sodium Tripolyphosphate |
|---|---|---|---|
| 5 | 25 | 1 | 10 |
| 6 | 16.7 | 1 | 6.7 |
| 7 | 12.5 | 1 | 5 |

TABLE 3
Results of particle size analysis and surface
potential analysis of samples 1-7.

| Samples | Average Particle Size (nm) | PDI* | Surface Potential (mV) |
|---|---|---|---|
| 1 | 111.7 ± 6.65 | 0.364 | 25.9 ± 1.12 |
| 2 | 118.1 ± 2.49 | 0.402 | 25.9 ± 0.37 |
| 3 | 127.6 ± 5.27 | 0.363 | 25.2 ± 0.55 |
| 4 | 147.2 ± 8.80 | 0.406 | 22.2 ± 0.24 |
| 5 | 97.0 ± 1.56 | 0.378 | 24.3 ± 0.45 |
| 6 | 112.1 ± 2.37 | 0.299 | 23.1 ± 0.55 |
| 7 | 129.9 ± 3.41 | 0.296 | 21.3 ± 0.80 |

*PDI: Polydispersity index

On the other hand, in anr example of the drug carrier of the disclosure, the same chitosan solution, alginate solution and sodium tripolyphosphate solution wre prepared. In addition, amoxicillin was dissolved in 0.01 N NaOH solution to achieve the concentration of 1.5 mg/ml and pH=7.4. According to steps S21 to S24, samples A-G were prepared by using the proportions listed in Tables 4-5 below. The obtained results of particle size analysis, surface potential analysis and active substance encapsulation rate analysis of samples A-G are shown in Table 6 below.

TABLE 4
Weight ratios of each components used in samples A-D.

| | | | Sodium | |
|---|---|---|---|---|
| Sample | Chitosan | Alginate | Tripolyphosphate | Amoxicillin |
| A | 25 | 1 | 10 | 30 |
| B | 16.7 | 1 | 6.7 | 20 |
| C | 12.5 | 1 | 5 | 15 |
| D | 8.3 | 1 | 3.3 | 10 |

TABLE 5
Weight ratios of each components used in samples E-G.

| | | | Sodium | |
|---|---|---|---|---|
| Sample | Chitosan | Polyacrylic Acid | Tripolyphosphate | Amoxicillin |
| E | 25 | 1 | 10 | 30 |
| F | 16.7 | 1 | 6.7 | 20 |
| G | 12.5 | 1 | 5 | 15 |

TABLE 6
Results of particle size analysis, surface potential analysis
and encapsulation rate analysis of samples A-G.

| | Average Particle | | Surface | Encapsulation |
|---|---|---|---|---|
| Samples | Size (nm) | PDI* | Potential (mV) | Rate (%) |
| A | 111.7 ± 3.01 | 0.373 | 24.4 ± 0.73 | 71.6 ± 11.1 |
| B | 119.2 ± 0.75 | 0.390 | 23.7 ± 0.50 | 74.5 ± 3.39 |
| C | 124.7 ± 1.85 | 0.340 | 23.3 ± 0.64 | 73.7 ± 2.96 |
| D | 153.2 ± 4.10 | 0.382 | 21.1 ± 0.93 | 75.4 ± 0.51 |
| E | 110.3 ± 4.26 | 0.381 | 23.4 ± 0.98 | 59.1 ± 0.56 |
| F | 118.5 ± 6.11 | 0.342 | 21.7 ± 0.48 | 59.1 ± 0.10 |
| G | 139.6 ± 1.91 | 0.261 | 21.1 ± 0.41 | 58.9 ± 1.17 |

*PDI: Polydispersity index

From the data listed in Tables 3 and 6, it can be seen that the carrier structures and drug carriers obtained in the above examples are all nanoparticles, which can be expected to exhibit excellent absorption efficiency in the organism. In addition, since the carrier structure and drug carrier of the disclosure are not core-shell structure, the method of the disclosure adopts solution method instead of water-in-oil emulsification method, that is, the carrier structure and drug carrier of the disclosure are prepared by evenly mixing the solution of each component and generating mutual electrostatic attraction by virtue of their respective electrification characteristics. Preparing by solution method not only has the advantages of simple operation, but also, according to the PDI data, the particle size of the prepared drug carrier has small distribution and good homogeneity.

In addition, to simulate the carrier structure and drug carrier of the disclosure in acidic environment, taking the sample A and E prepared above for example, sample A and E were put in an environment at pH 2.5, 4.0, 5.0, 6.0, and 7.4, in order to respectively represent the gastric acidic environment, different depths in gastric parietal mucosa layer, and gastric parietal cell layer. Then, a nanoparticle-size and potential analyzer (Zetasizer Nano ZS90) and a transmission electron microscope (TEM) were used to analyze and observe the changes in structural features.

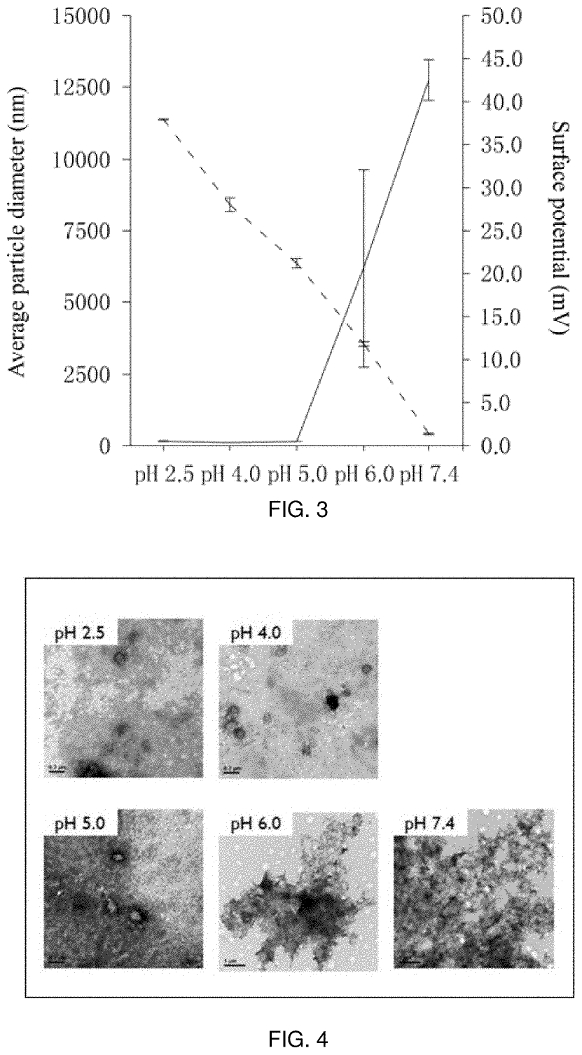

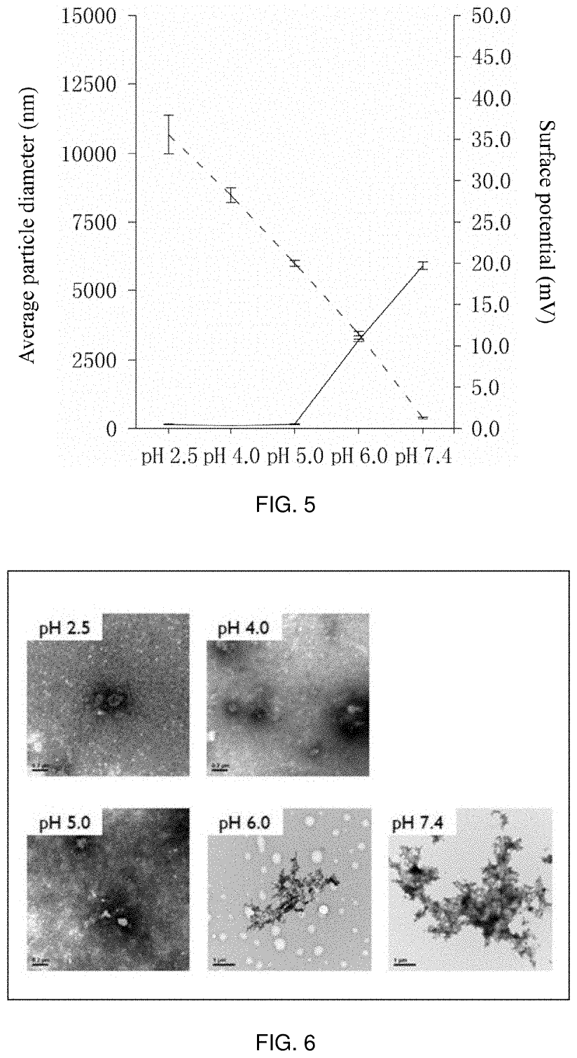

The analysis results are shown in FIGS. 3-6, wherein FIGS. 3-4 are the results of sample A, while FIGS. 5-6 are the results of sample E. In the gastric acidic environment with a pH value of 2.5, the nanostructure of the drug carrier of the sample A or sample E was not damaged by the erosion of gastric acid, and the surface still had a positive charge of 39 to 40 mV. Because the chitosan, alginate and polyacrylic acid contained in the drug carrier of the disclosure have the property of sticking to the mucosal tissue, the drug carrier tends to stick to the gastric parietal mucosa. The pH value of gastric parietal mucosa is about 4.0, 5.0 and 6.0 depending on its depth. In the attached figures, both samples A and E clearly showed that the nano-structures of the drug carriers still had stable particle size at pH=4.0 and 5.0, and the surface potential of the drug carriers were still between 20 and 30 mV. On the other hand, when the drug carriers were in the pH 6.0 environment of the deeper simulated gastric parietal mucosa or in the pH 7.4 environment of the gastric parietal cell layer, the chitosan becomes uncharged and the surface potential of the drug carriers tended to be 0 mV due to the about neutral pH value, and thus the nanostructure of the drug carrier become loose. In addition to the figures, the changes of nanostructures could also be observed from the TEM photos. In the environment having a pH value higher than 6.0, obvious aggregation phenomenon of the drug carriers appears, and the nanoparticle structure was difficult to be seen.

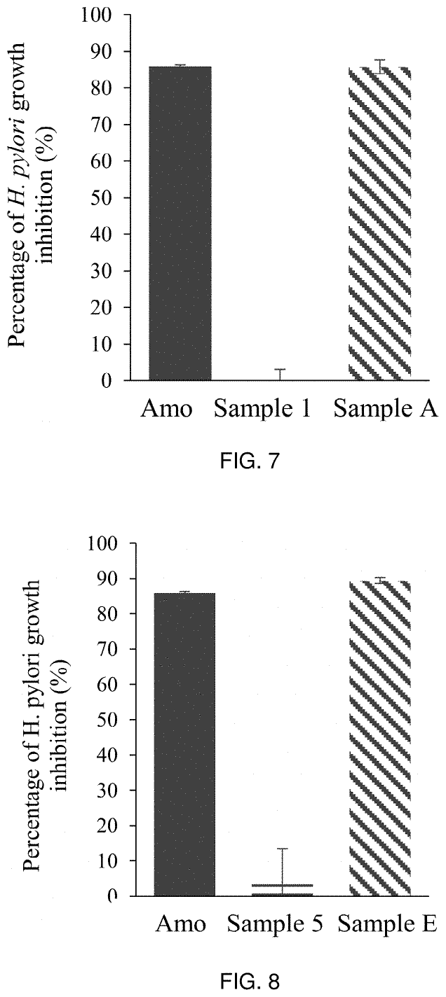

FIGS. 7 and 8 show the results of the in vitro application of the drug carrier according to some embodiments of this invention. In the in vitro experiment, a suspension of H. pylori was obtained and respectively added with amoxicillin (with a minimum inhibitory concentration of about 0.5 μg/ml and the concentration was fixed at 0.5 μg/ml), samples 1, 5, A and E above. Next, the suspensions of H. pylori, after the addition of amoxicillin and the above samples, were further cultured for 48 hours, the OD₄₅₀ of the suspension each was measured to determine the effect of inhibiting H. pylori. The experimental results are shown in FIG. 7. Because the active substances contained in samples A and E were amoxicillin, so the addition of samples A and E basically had the same inhibitory effect as the addition of amoxicillin. It could be observed from the experimental results that the carrier structure of sample 5 still has little ability to inhibit H. pylori even without any active substance. Through this test, it can be clearly seen that both the carrier structure and the drug carrier of the disclosure have the ability to inhibit H. pylori, especially the drug carrier with appropriate active substances has an obvious effect.

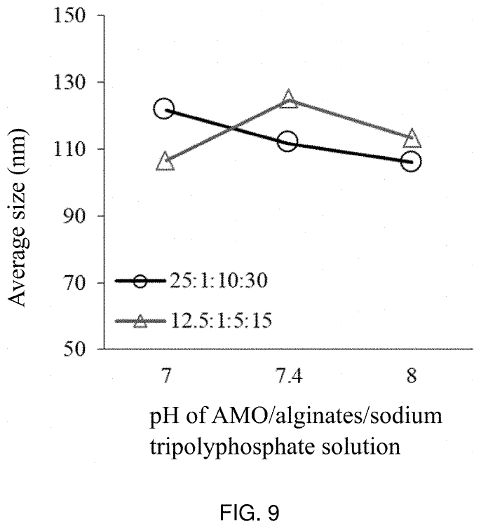

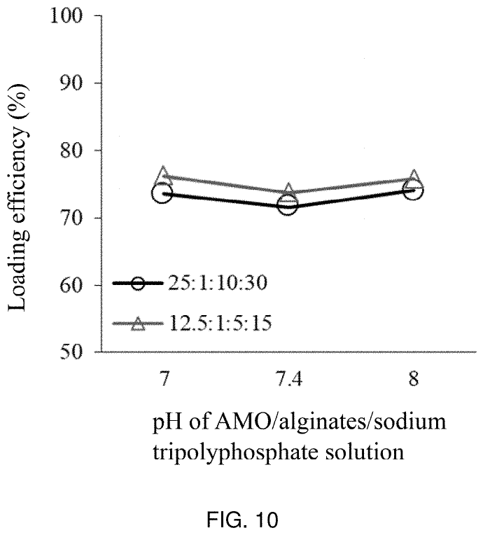

In addition, in an example of the drug carrier of this invention, after adjusting the pH value of the AMO/alginate/sodium tripolyphosphate solution to 7.0, 7.4, and 8.0, a chisonsolution (pH=4.0) was added to form nanoparticles containing amoxicillin. Subsequently, the particle size of the nanoparticles and the encapsulating rate of amoxicillin were measured. Please refer to FIGS. 9-10. FIG. 9 depicts a diagram of average particle size versus different pH values of AMO/alginates/soldium tripolyphosphare solution when the nanoparticiles were prepared with chitosan:alginate:sodium tripolyphosphate:amoxicillin weight ratios of 25:1:10:30 and 12.5:1:5:15. FIG. 10 depicts a diagram of encapsulating rate versus different pH values of AMO/alginates/soldium tripolyphosphare solution when the nanoparticiles were prepared with chitosan:alginate:sodium tripolyphosphate:amoxicillin having weight ratios of 25:1:10:30 and 12.5:1:5:15.

From the results of the particle size and the encapsulating rate data shown in FIG. 9-10, it can be known that when the weight ratio of chitosan:alginate:sodium tripolyphosphate:amoxicillin is 25:1:10:30, the particle size becomes smaller as the pH of the AMO/alginate/sodium tripolyphosphate solution increases. At the same time, the encapsulating rate decreases first and then increases as the pH value increases. When the weight ratio of chitosan:alginate:sodium tripolyphosphate:amoxicillin is 12.5:1:5:15, the particle size first increases and then decreases with the increase of pH value of the AMO/alginate/sodium tripolyphosphate solution, and the encapsulating rate first decreases and then increases with the increase of pH value.

Furthermore, the data results of FIGS. 9-10 show that when the weight ratio of chitosan:alginate:sodium tripolyphosphate:amoxicillin is 12.5:1:5:15, and the pH value of the AMO/alginate/sodium tripolyphosphate solution is 7.0 during preparation, the smallest nanoparticles (106.6 nm) and the highest amoxicillin encapsulating rate (76.2%) can be obtained.

In light of the foregoing, in this disclosure, when the drug carriers adhere to the mucosal tissue and close to the neutral environment of the gastric parietal cell layer, the nano-structure of the drug carrier gradually disintegrated due to the changes of the electrical properties of the chitosan and alginate or polyacrylic acid to release the active substance of the drug carrier. Such releasing property allows the drug to be released closer to the accumulation sites of pathogens, helping to improve the efficacy of the active substance.

The above embodiments express only a few embodiments of the disclosure, which are described in a more specific and detailed manner. However, it couldn't therefore be seen as the limitation to the claims. It should be noted that, for one with ordinary skills in the art, a number of adjustments and improvements could be made without deviating from the idea of the disclosure, which fall within the protection scope of the claims. Therefore, the protection scope of the patent of the disclosure shall be subject to the attached claims.

What is claimed is:

1. A method of preparing a carrier structure for carrying an active substance, comprising:
preparing an aqueous solution of a negatively charged polymer with a pH value of 6-8 by dissolving the negatively charged polymer in a NaOH aqueous solution;
preparing an aqueous solution of sodium tripolyphosphate with a pH value of 6-8 by dissolving the sodium tripolyphosphate in a NaOH aqueous solution;
preparing an aqueous solution of chitosan with a pH value of 3-5;
mixing 100 parts by weight of the aqueous solution of the negatively charged polymer, 330-1000 parts by weight of the aqueous solution of the sodium tripolyphosphate, and 830-2500 parts by weight of the aqueous solution of chitosan to form an initial mixture; and
reacting the initial mixture for 5-60 minutes to self-assemble the negatively charged polymer, the sodium tripolyphosphate, and the chitosan to form the carrier structure.

2. The method of claim 1, wherein the carrier structure has a diameter of 90-150 nm.

3. The method of claim 1, wherein the carrier structure in an aqueous solution has a surface potential of is 15-30 mV.

4. The method of claim 1, wherein the negatively charged polymer comprises alginate, heparin, polyacrylic acid, polystyrene sulfonate, poly(maleic acid), hyaluronic acid, or any combinations thereof.

5. A carrier structure for carrying a drug prepared by the method of claim 1.

6. A method of preparing a drug carrier carrying an active substance, the method comprising:
preparing an aqueous solution of a negatively charged polymer with a pH value of 6-8 by dissolving the negatively charged polymer in an aqueous solution of NaOH;
preparing an aqueous solution of sodium tripolyphosphate with a pH value of 6-8 by dissolving the sodium tripolyphosphate in an aqueous solution of NaOH;
preparing an aqueous solution of the active substance with a pH value of 6-8 by dissolving the active substance in an aqueous solution of NaOH;
mixing 100 parts by weight of the aqueous solution of the negatively charged polymer, 330-1000 parts by weight of the aqueous solution of the sodium tripolyphosphate, and 2000-3000 parts by weight of the aqueous solution of the active substance to form an initial mixture;
adding 830-2500 parts by weight of an aqueous solution of chitosan with a pH value of 3-5 into the initial mixture to form an active mixture; and
reacting the active mixture for 5-60 minutes to self-assemble the negatively charged polymer, the sodium tripolyphosphate, the active substance and the chitosan to form the drug carrier carrying the active substance.

7. The method of claim 6, wherein the drug carrier has a particle diameter of 110 to 160 nm.

8. The method of claim 6, wherein the drug carrier in an aqueous solution has a surface potential of 15-25 mV.

9. The method of claim 6, wherein the active substance comprises amoxicillin, clarithromycin, omeprazole, penicillin or any combinations thereof.

10. The method of claim 6, wherein the negatively charged polymer comprises alginate, heparin, polyacrylic acid, polystyrene sulfonate, poly(maleic acid), hyaluronic acid, or a combination thereof.

11. The method of claim 6, wherein an encapsulation rate of the active substance in the drug carrier is 55-75%.

12. The method of claim 6, wherein the drug carrier carrying the active substance contains 32-38 wt % of the active substance.

13. A drug carrier carrying an active substance prepared by the method of claim 6.

14. A method of treating a gastrointestinal disease, comprising:
applying an effective dosage of the drug carrier carrying an active substance of claim 13 to a host having a gastrointestinal disease caused by Helicobacter pylori, wherein the active substance comprises amoxicillin, clarithromycin, omeprazole, penicillin or any combinations thereof.

15. The method of claim 14, wherein the effective dosage is 1-10 mg/kg body weight per day.

16. The method of claim 14, wherein the host is a human.

17. The method of claim 14, wherein the gastrointestinal disease comprises chronic gastritis, duodenal ulcer, gastric ulcer, gastric lymphoma, gastric cancer, gastric mucosal atrophy, intestinal metaplasia or any combinations thereof.
